(19) United States
(12) Patent Application Publication
(10) Pub. No.: US 2004/0059412 A1
(43) Pub. Date: Mar. 25, 2004

(54) Heart valve holder

(76) Inventors: Thomas William Lytle IV, Round Rock, TX (US); Jimmie B. Allred III, Skaneateles, NY (US)

Correspondence Address: J. MIKE AMERSON, WILLIAMS, MORGAN & AMERSON, P.C., 10333 RICHMOND, SUITE 1100, HOUSTON, TX, 77042, US

(21) Appl. No.: 10/254,324

(22) Filed: Sep. 25, 2002

(51) Int. Cl. A61F 2/24; A61F 11/00

(52) U.S. Cl. 623/2.11; 606/108

(57) ABSTRACT

Several embodiments of a heart valve holder are described. In one instance, the heart valve holder is comprised of a plurality of hollow members having a plurality of openings formed therein. The heart valve holder is adapted to engage a heart valve through use of vacuum pressure. In another instance, a heart valve holder comprised of at least one inflatable balloon member is disclosed. When inflated, the balloon members may be used to hold the heart valve during implantation.

### BACKGROUND OF THE INVENTION

The present invention relates generally to medical devices, and, more particularly, to a heart valve holder that may be used by a surgeon during the process of replacing damaged or diseased heart valves.

The human heart includes four valved chambers (left and right atria and ventricles) for pumping blood through the body. Each ventricle has two valves to control the inflow of blood from the atria and the outflow of blood to the lungs (right ventricle) or to the rest of the body (left ventricle). In the case of the right ventricle, the inlet and outlet valves are the tricuspid and pulmonary valves, and in the case of the left ventricle they are the mitral and aortic valves. During each cycle of the heart's operation, the mitral and tricuspid valves open simultaneously to allow blood to flow into the ventricles while the aortic and pulmonary (outlet) valves are closed. The ventricles then contract, and the resulting blood pressure therein closes the mitral and tricuspid (inlet) valves while opening and forcing blood outward through the aortic and pulmonary valves.

In some individuals one or more of the foregoing valves may not function properly, usually as a result of disease-induced valve damage, degeneration or a congenital defect. In the case of the aortic valve, in particular, dysfunction often results from a narrowing of the valve orifice (stenosis), or from valve regurgitation such that the valve does not fully open or close. Severe heart valve dysfunction is life threatening. For the past several years, severe valve dysfunction has been treated by replacing the incompetent valve with a mechanical prosthesis, or alternatively, with a bioprosthetic valve (i.e., a valve comprising human or animal tissue). The terms “bioprosthetic valve” and “tissue valve” as used herein are synonymous and are used interchangeably. Tissue valves have the advantage of a lower incidence of blood clotting (thrombosis). Hence, patients receiving such a tissue valve, unlike those receiving a mechanical valve, do not usually require prolonged anticoagulation therapy with its potential complications and patient inconvenience.

Surgically-implanted heart valve prostheses have extended the life expectancy of many patients with defective natural heart valves. By way of example, an aortic prosthetic valve is implanted in the patient during a surgical procedure in which a segment of the aorta near the natural valve is slit open so that the malfunctioning leaflets can be cut out and the prosthetic valve is sutured within an intact segment of the aorta adjacent to the heart. The surgical procedure is exacting because of the difficulty of accessibly exposing the aorta for the surgeon. Accordingly, the valve itself lies in a relatively cramped space. Because of the crowded surgical field, holding the prosthesis in place while the surgeon places the sutures to attach it to the interior of the patient's aorta presents an especially challenging problem.

In the case of human aortic valve replacement, a commonly used tissue valve can be categorized as an allograft (usually an aortic valve from a human cadaver, sometimes referred to as a homograft). In addition, some human aortic valves have been replaced with pulmonary autografts; that is, a pulmonary valve from the same patient which in turn is then replaced with an allograft valve or tissue valve constructed from nonvalvular tissue (e.g., pericardium).

Xenografts (heart valves comprising tissue from a non-human donor animal) are also commonly used for human valve replacement. In particular, the porcine aortic valve is often used since it is similar in anatomy to the human aortic valve (both being trileaflet) and is readily available in a variety of sizes. The porcine aortic xenograft has been used for human valve replacement, both stented, i.e., mounted in a frame, and unstented, i.e., without a frame.

Unstented bioprosthetic valves require a more exacting surgical procedure for insertion into a patient than do stented valves. Correct valve selection, orientation, and sizing are important to avoid valve distortion and subsequent malfunction. Moreover, stentless tissue valve implantation is made even more difficult due to the nature of the product itself. Stentless tissue valves are very flexible and lack any significant structural rigidity. The absence of a fixed structure within the valve that can retain the valve in a desired position for suture attachment is a persistent problem in stentless valve implantation. Even routine handling and positioning of the valves is very difficult during the surgical procedure.

The presently used technique of implanting an unstented xenograft or allograft tissue valve requires holding the flaccid valve between the fingers and estimating the appropriate suture placement relative to the diseased aortic root. Such a technique complicates the insertion procedure and frequently results in geometric mismatch of the replacement valve with the recipient's native aortic root. Due to the foregoing difficulties, many surgeons currently prefer to implant stented bioprosthetic valves even though unstented valves, both xenografts and allografts, minimize turbulence and should therefore reduce thrombosis and embolism in comparison to stented valves. Moreover, due to the cramped working area, protection of the valve leaflets during the surgical procedure is also important. That is, it is very desirable to reduce the risk of damaging the heart valve leaflets during surgery.

The present invention is directed to a method and system to solve, or at least reduce, some or all of the aforementioned problems.

### SUMMARY OF THE INVENTION

The present application is directed to various embodiments of a bioprosthetic heart valve holder. In one illustrative embodiment, a heart valve holder in accordance with the present invention comprises a housing, a plurality of hollow members coupled to the housing, each of the plurality of hollow members having a plurality of openings formed therein, and an inlet port coupled to the housing, the inlet port coupled to a vacuum source whereby a vacuum pressure may be supplied via the inlet port to the openings in the hollow members. As used herein, the term and “vacuum pressure” refers to a pressure less than the ambient air pressure adjacent to a prosthetic heart valve. By engaging the openings in the hollow members directly to a portion of a bioprosthetic heart valve, particularly to a tissue component of such a valve, a suction force may be applied to retain the valve on the holder. Specifically, the hollow members are positioned adjacent to a heart valve, and the vacuum pressure creates a suction against the valve which is used to secure the valve to the heart valve holder. By employing a relatively low vacuum pressure, a relatively strong suction force may be developed because of the greater pressure differential between the vacuum pressure and the ambient pressure. Correspondingly, a relatively high vacuum pressure may be used to employ a relatively weaker suction force upon the valve.

In a particular illustrative embodiment, the plurality of openings in the hollow members are formed in an exterior surface of the hollow members, and the hollow members are adapted to engage, preferably by direct contact, an interior surface of a heart valve. In another embodiment, the plurality of openings on the hollow members are formed on an interior surface of the hollow members, and the hollow members are adapted to engage an external surface of a heart valve.

In other illustrative embodiments, the heart valve holder employs at least one balloon-type structure to assist in holding the heart valve during implantation. In one embodiment, the heart valve holder comprises a housing, at least one inlet port coupled to the housing, and at least one balloon member coupled to the housing and in fluid communication with the inlet port, the balloon member adapted to engage at least a portion of an interior surface of a heart valve when a fluid is supplied to the balloon member via the inlet port.

In a particular embodiment, the holder comprises a balloon member positioned around an exterior surface of the housing, sealingly coupled to the housing, and in fluid communication with the inlet port, whereby the balloon member is adapted to be inflated by introduction of a fluid through the inlet port and the openings. In yet another illustrative embodiment, the heart valve holder comprises a balloon member sealingly coupled to a bottom surface of the housing and in fluid communication with the inlet port, the balloon member having at least three portions, each of which are adapted to engage, when inflated, at least a portion of a sinus of Valsalva in a heart valve.

In a further embodiment, the heart valve holder comprises three balloon members, each of which are operatively coupled to and in fluid communication with at least one inlet port, each of the balloon members adapted to engage, when inflated, at least a portion of a sinus of Valsalva in a heart valve and one another.

### BRIEF DESCRIPTION OF THE DRAWINGS

The invention may be understood by reference to the following description taken in conjunction with the accompanying drawings, in which like reference numerals identify like elements, and in which:

FIGS. 1A-1C depict one illustrative embodiment of the present invention;

FIGS. 2A-2D depict another illustrative embodiment of the present invention; and

FIGS. 3A-3B depict yet another illustrative embodiment of the present invention.

While the invention is susceptible to various modifications and alternative forms, specific embodiments thereof are provided in the drawings and described herein in detail. It should be understood, however, that the description of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. In the development of any such actual embodiment, numerous implementation-specific decisions must be made to achieve the developers' specific goals, such as compliance with health-related (or human-related), system-related and business-related constraints, which will vary from one implementation to another. While such a development effort might be complex and time-consuming, it is nevertheless a routine undertaking for those of skill in the art having the benefit of the present disclosure.

Although the various regions and structures of the heart are depicted in the drawings as having very precise, sharp configurations and profiles, those skilled in the art recognize that, in reality, these regions and structures are not as precise as indicated in the drawings. Additionally, the relative sizes of the various features and structures depicted in the drawings may be exaggerated or reduced as compared to the size of those features or structures on real-world devices. Nevertheless, the attached drawings are included to describe and explain illustrative examples of the present invention.

In general, the present invention is directed to various embodiments of a heart valve holder. The valve holders described herein may be used to hold a variety of different heart valves, and they may be used in connection with a variety of different surgical procedures, e.g., full-root, root-inclusion, and complete and modified sub-coronary procedures, etc. Thus, neither the specific type of valve used nor the type of surgical procedure performed should be considered a limitation of the present invention unless such limitations are clearly set forth in the appended claims.

Further, as will be recognized by those skilled in the art after a complete reading of the present application, the devices disclosed herein may be employed by a variety of different materials and techniques. For example, at least a portion of the device may be supplied with the heart valve provided to a surgeon. Alternatively, in practice, the inventions disclosed herein may be essentially instruments that are used in the heart valve installation procedure.

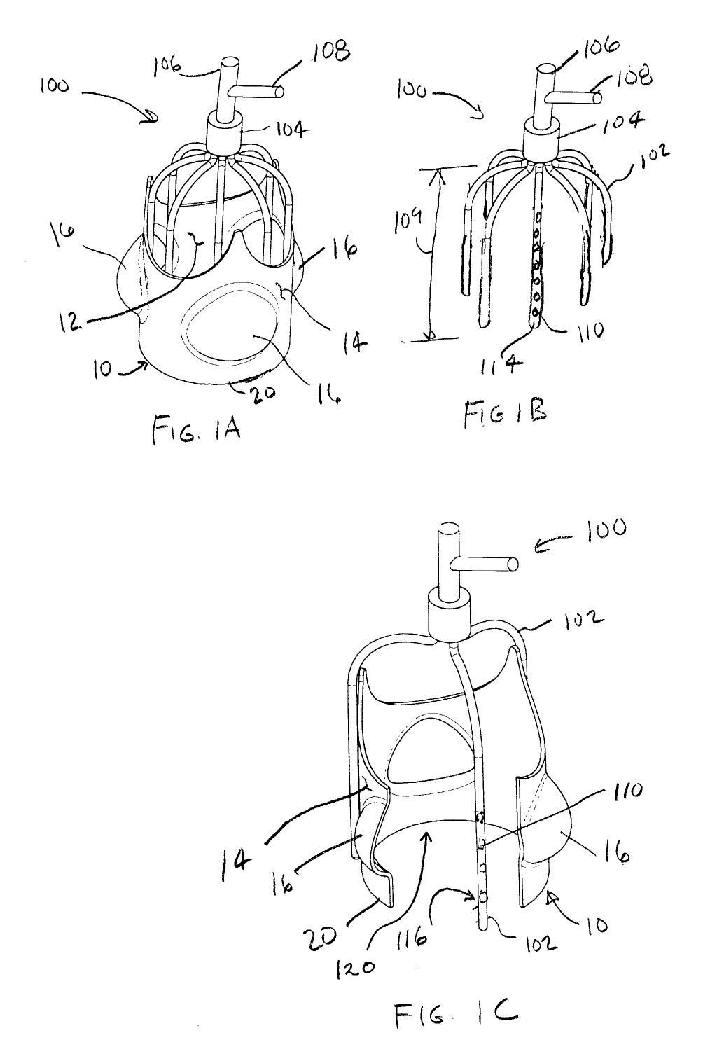

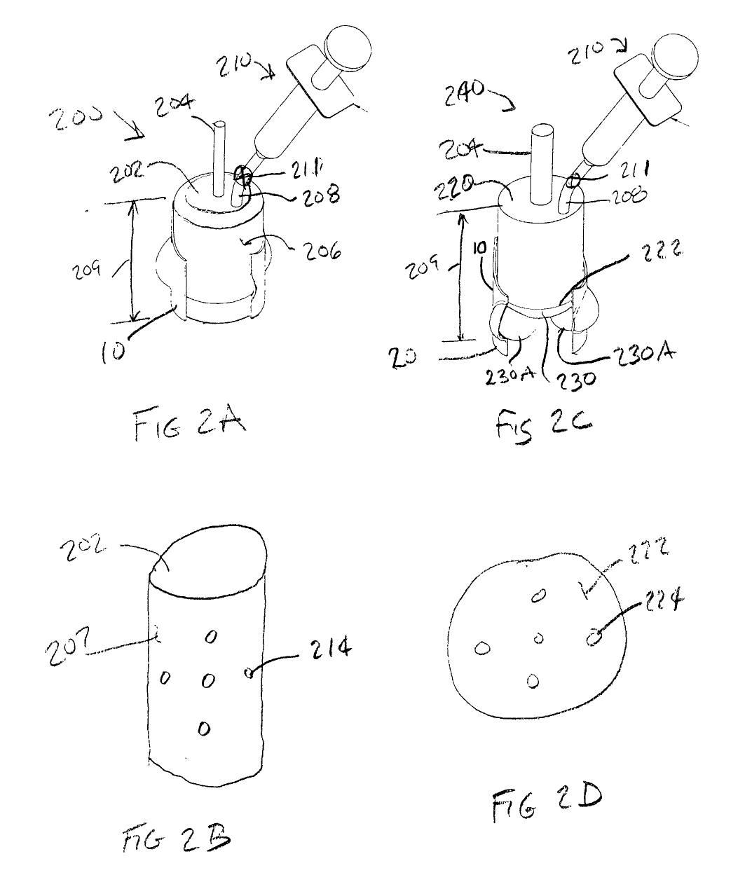

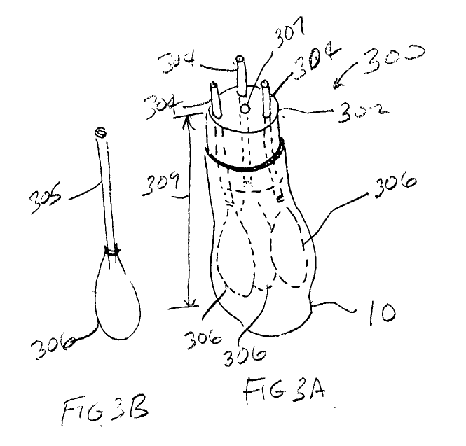

In general, the present application is directed to a variety of heart valve holders. More generally, the embodiments depicted in FIGS. 1A-1C are directed to a holder that involves the use of vacuum suction to couple the heart valve to the holder, while the embodiments depicted in FIGS. 2A-2D and 3A-3B involve the use of inflatable balloon-type structures to accomplish the same task. The details of each embodiment will be described further below.

FIGS. 1A-1C depict a valve holder 100 adapted to hold a stentless valve 10 through use of vacuum pressure. In one embodiment (see FIGS. 1A-1B), the valve holder 100 is adapted to be positioned on the inside of the stentless valve 10, i.e., to engage an inner or blood contacting surface of the valve. In another embodiment (see FIG. 1C), holder 100 is adapted to be positioned on the outside of the stentless valve 10. Alternative embodiments (not shown) using both inside and outside engagement members, are also contemplated.

As shown in FIGS. 1A-1B, the valve holder 100 is comprised of a plurality of hollow members 102 that are coupled to a housing 104. Each of the hollow members 102 has a plurality of openings 110 formed therein (only depicted on one hollow member 102 for purposes of clarity). The housing 104 has a structural member 106 that is adapted to be coupled to a handle (not shown) by a variety of known techniques. For example, the structural member 106 may have a threaded male connection that is adapted to engage a corresponding female threaded connection in the end of a handle. The housing 104 further comprises a vacuum port 108. The housing 104 and hollow members 102 are arranged and configured such that the hollow members 102 are in fluid communication with the vacuum port 108. Although not depicted in the drawings, a shut-off valve may also be positioned on the vacuum port 108. The hollow members 102 and openings 110 may be considered to constitute a conduit means that allow a vacuum pressure to be applied to a heart valve to be implanted in a patient. Depending upon the physical configuration of the conduit means, the vacuum pressure may be applied to an interior or an exterior surface of a heart valve.

The valve holder 100 may be manufactured in a variety of configurations, and it may be made from a variety of materials. For example, the hollow members 102 may be made from a variety of materials, e.g., plastic, stainless steel, etc., and the number of hollow members 102 may vary. In the embodiment depicted in FIGS. 1A-1B, the heart valve holder 100 is comprised of eight hollow members 102, although more or fewer could be used. The hollow members 102 may be manufactured from structural members such as round tubing, square tubing, etc. Moreover, the axial length 109 of the heart valve holder 100 may also vary, e.g., the length 109 may range from approximately one (1) to three (3) inches.

The number, size and configuration of the openings 110 on the hollow members 102 may vary. For example, the openings 110 may have a circular, elliptical, or rectangular configuration. Moreover, the number and location of the openings 110 need not be uniform on each of the hollow members 102. For example, the openings 110 may be spaced apart by a distance of approximately 0.1-0.125 inch. In one particularly illustrative embodiment, the hollow members 102 are comprised of hollow stainless steel tubes having an outer diameter of approximately 0.05″, and there are approximately 6-8 openings 110 spaced apart approximately 0.125″ on each of the hollow members 102. In the embodiment depicted in FIGS. 1A-1B, the openings 110 are positioned on an exterior surface 114, i.e., an outwardly facing surface, of the hollow members 102 such that the openings 110 may engage an interior surface 12 of the stentless valve 10.

In the embodiment depicted in FIG. 1C, the hollow members 102 are positioned so as to engage an exterior surface 14 of the stentless heart valve 10. In the embodiment depicted in FIG. 1C, the openings 110 are positioned on an interior surface 116, i.e., an inwardly facing surface, of the hollow member 102 such that they may engage an exterior surface 14 of the stentless heart valve 10. For purposes of clarity, only three hollow members 102 of the valve holder 100 are depicted in FIG. 1C. Moreover, only four representative openings 110 (with use of hidden lines) formed in one of the hollow members 102 are depicted in FIG. 1C, although more or fewer openings can be used. The number of hollow members 102 used on the valve holder 100 may vary depending upon whether it is adapted to engage the interior surface 12 or exterior surface 14 of the heart valve 10. For example, if the heart valve holder 100 is to be used to engage the exterior 14 of the heart valve 10, fewer hollow members 102 may be used so as to avoid the external protrusions 16 associated with the sinuses of Valsalva in the heart valve 10. For example, a heart valve holder 100 may be comprised of 3-6 such hollow members 102. Additionally, the valve holder 100 may be sized for a unique size of heart valve 10.

In operation, an access opening or slit may be cut in the aorta. Thereafter, the diseased or damaged heart valve (not shown) may be removed. After the surgeon has confirmed the appropriate size of the replacement stentless heart valve 10, the heart valve 10 may be positioned around the valve holder 100, depicted in FIGS. 1A-1B, or it may be positioned within the interior region 120 defined by the hollow members 102 of the valve holder 100 depicted in FIG. 1C. Thereafter, vacuum pressure from a source within the operating room may be applied to the vacuum port 108 and to the openings 110. The stentless heart valve 10 may then be moved or adjusted as desired by the surgeon. In some situations, the vacuum supply may be regulated such that a relatively weak vacuum pressure is employed when positioning the heart valve 10 on the heart valve holder 100, and a relatively stronger vacuum pressure is supplied after the heart valve 10 is correctly positioned on the valve holder 100 by the surgeon.

Once the heart valve 10 is properly positioned on the heart valve holder 100, a handle (not shown) may be secured to the structural member 106 of the housing 104. The heart valve 10 may then be properly positioned in the heart, and the surgeon may use a plurality of stitches to secure the distal end 20 of the heart valve 10 in position within the heart. Thereafter, the vacuum pressure may be released, the valve holder 100 withdrawn, and the surgeon may complete the installation of the stentless heart valve 10. Alternatively, the surgeon may also secure the proximal end of the valve before releasing the vacuum pressure and removing the valve.

In general, the valve holders depicted in FIGS. 2A-2D involve the use of a balloon-type member to hold the stentless heart valve 10. As shown in FIGS. 2A-2B, one embodiment of the heart valve holder 200 is comprised of a cylindrical housing 202, a structural member 204, a balloon member 206 and a fluid inlet 208 having a valve 211 formed thereon. The housing 202 and balloon member 206 are configured such that the balloon member 206 is in fluid communication with the fluid inlet 208. The balloon member 206 is positioned around an exterior surface 207 of the housing 202 and sealingly engaged or coupled to the housing 202. Such a sealing engagement may be accomplished by use of an adhesive, or other known techniques. The balloon member 206 may be comprised of a variety of materials, such as a silicone rubber material. Also depicted in FIG. 2A is a syringe 210 that, in one embodiment, may be used to inject a fluid, such as air or saline into the housing 202 so as to inflate the balloon member 206, as described more fully below.

The housing 202 has a plurality of openings 214 formed around the circumference of the housing 202, as indicated in FIG. 2B. However, in some situations, only a single opening 214 may be formed in the housing 202. As described more fully below, a fluid, such as saline or air, may be supplied via the fluid inlet 208 to the housing 202 and, thereafter, to the balloon member 206 via the openings 214 in the housing 202. In this manner, the balloon may be inflated when desired.

The member 204 may be connected to a handle (not shown) after the heart valve 10 is positioned around the heart valve holder 200. Alternatively, such a handle member may be directly coupled to the housing 202 by means of a threaded recess (not shown) formed in the housing 202. The housing 202 may be manufactured from plastic, stainless steel or other like material. Moreover, the size, shape and configuration of the housing 202 may be varied as a matter of design choice. Typically, the housing 202 and balloon member 206 are sized such that, when the balloon member 206 is deflated, there will be approximately ⅛″ clearance between the interior surface 12 of the stentless heart valve 10 and the balloon member 206. In practice, the valve holder 200 may be sized such that it is only useful for one particular size heart valve 10. Moreover, the length 209 of the heart valve holder 200 may vary, e.g., from approximately 1-3 inches.

In the embodiment depicted in FIGS. 2C and 2D, a balloon member 230 is positioned around a bottom surface 222 of the housing 220 of the valve holder 240. The balloon member 230 is sealingly engaged or coupled to the housing 220. Similar to the previous embodiment, the housing 220 and the balloon member 230 are configured such that the balloon member is in fluid communication with the fluid inlet 208. In this embodiment, the balloon member 230 has three portions 230A (only two of which are shown) that are configured such that, when inflated, the balloon portions 230A will substantially fill or nest within the sinuses of Valsalva in the heart valve 10. Fluid, e.g., saline or air, introduced via the fluid inlet 208 is used to inflate the balloon member 230 via a plurality of openings 224 formed in a bottom surface 222 of the housing 220. Alternatively, only a single opening 224 may be formed in the bottom surface 222 of the housing 220. The balloon members may be comprised of a variety of materials, such as a thin elastomer or silicone. Moreover, the balloon members 206, 230 may be considered to constitute inflatable means that, when inflated, are adapted to engage at least a portion of an interior surface of a heart valve.

In operation, as with the previous embodiments, after the surgeon confirms the proper size of the heart valve 10 to be implanted, the heart valve 10 may be positioned around the heart valve holder 200, 240, and fluid may be introduced into the housing 202, 220 through the fluid inlet 208. In one embodiment, the syringe 210 may be used to introduce such fluid. In other embodiments, the fluid may be introduced from another source, such as an air supply source within a hospital operating suite. In a specific embodiment, the fluid inlet 208 is provided with the valve 211 such that, after the balloon members 206, 230 are inflated, the valve 211 may be closed, thereby insuring that the balloons 206, 230 do not deflate until such time as desired by the surgeon. Similar to the situation discussed before, the balloon members 206, 230 may be slightly inflated during the process of positioning the heart valve 10 around the valve holder 200, 240. Thereafter, when proper positioning is confirmed by the surgeon, the fluid pressure within the housing 202, 220 may be increased to fully inflate the balloon members 206, 230, thereby securing the heart valve 10 to the valve holder 200, 240. Thereafter, a handle may be attached to the heart valve holder 200, 220 and the heart may be positioned into the proper location in the heart, the distal end 20 of the heart valve 10 may be secured in place by stitching, and the pressure within the balloon members 206, 230 may be released and the installation may be completed by the surgeon. Alternatively, both the proximal and distal ends may be secured before releasing the balloon pressure.

In the embodiment depicted in FIGS. 3A-3B, a plurality of balloon members are also employed as a mechanism for holding the stentless heart valve 10 in position. More particularly, as shown therein, the holder 300 is comprised of a housing 302, a plurality of fluid inlets 304 and three balloon members 306. The balloon members 306 are coupled to the fluid inlets 304 via a tube 305, as indicated in FIG. 3B. The length 309 of the heart valve holder 300 may vary, e.g., from approximately 1-3 inches. As before, the housing 302 may be comprised of a variety of materials, such as a plastic or stainless steel material, and the balloon members 306 may be made of silicone or a plastic material. In one embodiment, the tubes 305 are comprised of silicone rubber, and the balloon members 306 may be coupled to the tubes 305 by a variety of techniques, such as wrapping or gluing.

Fluid, such as saline or air, may be introduced into the balloon members 306 via the fluid ports 304. Although not depicted in the drawing, each of the fluid inlets 304 may be provided with a valve such that fluid flow to each of the balloon members 306 may be individually controlled. Alternatively, all of the fluid ports may be coupled to a single manifold from which the balloons are collectively inflated or deflated. When inflated, the balloon members 306 are sized and adapted to nest within the sinuses of Valsalva in the heart valve 10, and push against one another, thereby providing a means to securely position the heart valve 10 as required by the surgeon. As before, once the balloon members 306 are inflated, the heart valve 10 may be positioned within the heart, and the surgeon may stitch the proximal and/or distal ends of the heart valve 10 in position. Thereafter, the fluid pressure within the balloon members 306 may be reduced by opening the valves (not shown) coupled to the fluid inlets 304, and the holder may be withdrawn.

The present application is directed to various embodiments of a heart valve holder. In one illustrative embodiment, a heart valve holder in accordance with the present invention is comprised of a housing, a plurality of hollow members coupled to the housing, each of the plurality of hollow members having a plurality of openings formed therein, and an inlet port coupled to the housing, the inlet port adapted to be coupled to a vacuum source whereby a vacuum pressure may be supplied via the inlet port to the openings in the hollow members. In further illustrative embodiments, the plurality of openings in the hollow members are formed in an exterior surface of the hollow members, and the hollow members are adapted to engage an interior surface of a heart valve. In another embodiment, the plurality of openings on the hollow members are formed on an interior surface of the hollow members, and the hollow members are adapted to engage an external surface of a heart valve.

In other illustrative embodiments, the heart valve holder employs balloon-type structures to assist in holding the heart valve during implantation. In one embodiment, the heart valve holder is comprised of a housing, at least one inlet port coupled to the housing, and at least one balloon member sealingly coupled to the housing and in fluid communication with the inlet port, the balloon member adapted to engage at least a portion of an interior surface of a heart valve when a fluid is supplied to the balloon member via the inlet port. In a more detailed embodiment, the holder is comprised of a balloon member that is positioned around an exterior surface of the housing and sealingly engaged with the housing and in fluid communication with the inlet port, whereby the balloon member is adapted to be inflated by introduction of a fluid through the inlet port and the openings. In yet another illustrative embodiment, a heart valve holder is comprised of a balloon member that is positioned around a bottom surface of the housing and sealingly engaged with the housing and in fluid communication with the inlet port, the balloon member having at least three portions, each of which are adapted to engage, when inflated, at least a portion of a sinus of Valsalva in a heart valve. In a further embodiment, the heart valve holder is comprised of three balloon members, each of which are operatively coupled to and in fluid communication with at least one of the inlet ports, each of the balloon members adapted to engage, when inflated, at least a portion of a sinus of Valsalva of a heart valve and one another.

The particular embodiments disclosed above are illustrative only, as the invention may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A heart valve holder, comprising:
a housing;
a plurality of hollow members coupled to said housing, each of said plurality of hollow members having a plurality of openings formed therein; and
an inlet port coupled to said housing, said inlet port adapted to be coupled to a vacuum source whereby a vacuum pressure may be supplied via said inlet port to said openings in said hollow members.

2. The heart valve holder of claim 1, further comprising a valve coupled to said inlet port.

3. The heart valve holder of claim 1, wherein said plurality of openings are positioned on an exterior surface of said hollow members, and said hollow members are adapted to engage an interior surface of a heart valve.

4. The heart valve holder of claim 1, wherein said plurality of openings are positioned on an interior surface of said hollow members, and said hollow members are adapted to engage an exterior surface of a heart valve.

5. The heart valve holder of claim 1, wherein said plurality of hollow members comprises eight hollow members, and wherein said plurality of openings are formed on an external surface of each of said eight hollow members.

6. The heart valve holder of claim 1, wherein said plurality of hollow members comprises six hollow members and wherein said plurality of openings are formed on an interior surface of each of said six hollow members.

7. The heart valve holder of claim 1, wherein said housing is manufactured from a material comprised of at least one of a plastic and a stainless steel.

8. The heart valve holder of claim 1, wherein said plurality of hollow members are manufactured from a material comprised of at least one of a plastic and a stainless steel.

9. The heart valve holder of claim 1, wherein said plurality of hollow members are comprised of at least one of round tubing and square tubing.

10. The heart valve holder of claim 1, wherein said plurality of openings are comprised of at least one of circular openings, elliptical openings and rectangular openings.

11. The heart valve holder of claim 1, wherein said plurality of openings on said hollow members are spaced apart approximately 0.1-0.2 inch.

12. The heart valve holder of claim 1, further comprising a means for operatively coupling a handle to said housing.

13. A heart valve holder, comprising:
a housing;
a plurality of hollow members coupled to said housing, each of said plurality of hollow members having an exterior surface and a plurality of openings formed in said exterior surface; and
an inlet port coupled to said housing, said inlet port adapted to be coupled to a vacuum source whereby a vacuum pressure may be supplied via said inlet port to said openings in said hollow members.

14. The heart valve holder of claim 13, further comprising a valve coupled to said inlet port.

15. The heart valve holder of claim 13, wherein said hollow members are adapted to engage an interior surface of a heart valve.

16. The heart valve holder of claim 13, wherein said plurality of hollow members comprises eight hollow members.

17. The heart valve holder of claim 13, wherein said housing is manufactured from a material comprised of at least one of a plastic and a stainless steel.

18. The heart valve holder of claim 13, wherein said plurality of hollow members are manufactured from a material comprised of at least one of a plastic and a stainless steel.

19. The heart valve holder of claim 13, wherein said plurality of hollow members are comprised of at least one of round tubing and square tubing.

20. The heart valve holder of claim 13, wherein said plurality of openings are comprised of at least one of circular openings, elliptical openings and rectangular openings.

21. The heart valve holder of claim 13, wherein said plurality of openings on said hollow members are spaced apart approximately 0.1-0.2 inch.

22. The heart valve holder of claim 13, further comprising a means for operatively coupling a handle to said housing.

23. A heart valve holder, comprising:
a housing;
a plurality of hollow members coupled to said housing, each of said plurality of hollow members having an interior surface and a plurality of openings formed in said interior surface; and
an inlet port coupled to said housing, said inlet port adapted to be coupled to a vacuum source whereby a vacuum pressure may be supplied via said inlet port to said openings in said hollow members.

24. The heart valve holder of claim 23, further comprising a valve coupled to said inlet port.

25. The heart valve holder of claim 24, wherein said hollow members are adapted to engage an exterior surface of a heart valve.

26. The heart valve holder of claim 23, wherein said plurality of hollow members comprises six hollow members.

27. The heart valve holder of claim 23, wherein said plurality of hollow members are comprised of at least one of round tubing and square tubing.

28. The heart valve holder of claim 23, wherein said plurality of openings are comprised of at least one of circular openings, elliptical openings and rectangular openings.

29. The heart valve holder of claim 23, wherein said plurality of openings on said hollow members are spaced apart approximately 0.1-0.2 inch.

30. The heart valve holder of claim 23, further comprising a means for operatively coupling a handle to said housing.

31. A heart valve holder, comprising:
a housing;
at least one inlet port coupled to said housing; and
at least one balloon member sealingly coupled to said housing and in fluid communication with said at least one inlet port, said balloon member adapted to engage at least a portion of an interior surface of a heart valve when a fluid is supplied to said at least one balloon member via said at least one inlet port.

32. The heart valve holder of claim 31, wherein said housing is manufactured from a material comprised of at least one of a plastic and a stainless steel.

33. The heart valve holder of claim 31, wherein said at least one balloon member is positioned around an exterior surface of said housing, said at least one balloon member adapted to, when inflated, engage at least a portion of a generally cylindrical interior surface of a heart valve.

34. The heart valve holder of claim 31, wherein said housing has at least one opening formed therein, said at least one opening adapted to provide fluid communication between said inlet port and said at least one balloon member.

35. The heart valve holder of claim 31, wherein said housing has a bottom surface and wherein said at least one balloon member is positioned over said bottom surface of said housing, and at least a portion of said at least one balloon member is adapted to, when inflated, engage at least a portion of at least one of a plurality of the sinuses of Valsalva within a heart valve.

36. The heart valve holder of claim 35, wherein said bottom surface of said housing has at least one opening formed therein, said at least one opening adapted to provide fluid communication between said inlet port and said at least one balloon member.

37. The heart valve holder of claim 31, wherein said at least one balloon member is comprised of three balloon members, each of said three balloon members adapted to engage, when inflated, at least a portion of a Valsalva sinus within a heart valve.

38. The heart valve holder of claim 37, wherein said at least one inlet port is comprised of at least three inlet ports coupled to said housing, each of said inlet ports being dedicated to one of said three balloon members.

39. The heart valve holder of claim 31, wherein said at least one balloon member is manufactured from a material comprised of an elastomer selected from the group consisting of a rubber and a plastic.

40. The heart valve holder of claim 31, wherein said at least one balloon member is adapted to be inflated by introduction of at least one of saline and air introduced through said at least one inlet port.

41. A heart valve holder, comprising:
a housing;
at least one inlet port coupled to said housing; and
inflatable means for engaging at least a portion of an interior surface of a heart valve, said inflatable means being sealingly coupled to said housing and in fluid communication with said at least one inlet port.

42. The heart valve holder of claim 41, wherein said inflatable means comprises at least one balloon member sealingly coupled to said housing.

43. The heart valve holder of claim 41, wherein said housing has a bottom surface and wherein said inflatable means is positioned over said bottom surface of said housing, and at least a portion of said inflatable means is adapted to, when inflated, engage at least a portion of at least one of a plurality of sinuses of Valsalva within a heart valve.

44. The heart valve holder of claim 41, wherein said inflatable means is comprised of three balloon members, each of said three balloon members adapted to engage, when inflated, at least a portion of a sinus of Valsalva within a heart valve.

45. The heart valve holder of claim 41, wherein said inflatable means is adapted to be inflated by introduction of at least one of saline and air introduced through said at least one inlet port.

46. A heart valve holder, comprising:
a housing, said housing having an exterior surface and at least one opening formed in said exterior surface of said housing;
an inlet port coupled to said housing; and
a balloon member positioned around said exterior surface of said housing and sealingly engaged with said housing and in fluid communication with said inlet port, whereby said balloon member is adapted to be inflated by introduction of a fluid through said inlet port and said at least one opening.

47. The heart valve holder of claim 46, wherein said housing has a generally cylindrical configuration.

48. The heart valve holder of claim 46, wherein said balloon member is adapted to be inflated by introduction of a fluid comprised of at least one of saline and air through said inlet port.

49. A heart valve holder, comprising:
a housing, said housing having a bottom surface having at least one opening formed therein;
an inlet port coupled to said housing; and
a balloon member positioned around said bottom surface of said housing and sealingly engaged with said housing and in fluid communication with said inlet port, said balloon member having at least three portions, each of which is adapted to engage, when inflated, at least a portion of a Valsalva sinus in a heart valve.

50. The heart valve holder of claim 49, further comprising a plurality of openings in said bottom surface of said housing.

51. The heart valve holder of claim 49, wherein said at least three portions of said balloon member have a rounded exterior surface adapted to engage, when inflated, at least a portion of a sinus of Valsalva of a heart valve.

52. A heart valve holder, comprising:
a housing;
a plurality of inlet ports coupled to said housing; and
three balloon members, each of which are operatively coupled to and in fluid communication with at least one of said inlet ports, each of said balloon members adapted to engage, when inflated, at least a portion of a Valsalva sinus of a heart valve.

53. The heart valve holder of claim 52, wherein said three balloon members are adapted to be inflated by introduction of a fluid comprised of at least one of saline and air via said inlet ports.
